(19) United States
(12) Patent Application Publication
(10) Pub. No.: US 2009/0029925 A1
(43) Pub. Date: Jan. 29, 2009

(54) INTERACTION OF BIM WITH TRIM2, AN E3 UBIQUITIN LIGASE

(75) Inventor: Robert Meller, Portland, OR (US)

Correspondence Address: Stanley M. Hollenberg;Kolisch Hartwell, P.C., 200 Pacific Building, 520 S.W. Yamhill Street, Portland, OR, 97204, US

(73) Assignee: LEGACY EMANUEL HOSPITAL & HEALTH CENTER, Portland, OR (US)

(21) Appl. No.: 12/178,491

(22) Filed: Jul. 23, 2008

Related U.S. Application Data
(60) Provisional application No. 60/961,848, filed on Jul. 23, 2007.

(51) Int. Cl. A61K 38/07; C12Q 1/48; G01N 33/53; A61P 35/00; G01N 33/68

(52) U.S. Cl. 514/18; 435/15; 435/7.2; 435/7.8

(57) ABSTRACT

Methods, compositions, and cells for drug screening based on interaction between (1) a Bim polypeptide and/or an E2 polypeptide and (2) a TRIM2 polypeptide, which is identical to or corresponds to a Bim-selective E3 ubiquitin ligase. Methods and compositions for treating cancer based on levels of Bim protein, TRIM2 protein, and active MAPK protein are also provided.

### CROSS-REFERENCE TO PRIORITY APPLICATION

This application is based upon and claims the benefit under 35 U.S.C. § 119(e) of U.S. Provisional Patent Application Ser. No. 60/961,848, filed Jul. 23, 2007, which is incorporated herein by reference in its entirety for all purposes.

### BACKGROUND

Apoptosis is a form of cell death mediated by an intracellular program. The intracellular program proceeds through a series of biochemical events that result in various changes to cell morphology, including blebbing, cell shrinkage, nuclear fragmentation, chromatin condensation, and DNA fragmentation. Proteolytic enzymes termed caspases are thought to be primarily responsible for these hallmarks of apoptosis.

Apoptosis is fundamental to various biological processes, such as adult homeostasis (i.e., to keep the number of cells relatively constant), response to injury (i.e., to kill damaged cells), development (e.g., to help pattern developing tissues), and maturation of the immune system (e.g., to remove lymphocytes that are ineffective and/or harmful), among others. Accordingly, many diseases are associated with a change in the rate of apoptosis. For example, tumor cells may have a resistance to apoptosis that provides a selective growth advantage over normal cells. In contrast, unwanted apoptosis may cause the neurological damage prevalent in various neurological diseases, such as stroke, Parkinson's disease, and Alzheimer's disease.

Members of the Bcl-2 family regulate activation of the caspases. This family consists of two groups, namely, pro-death proteins (e.g., Bax) and pro-survival proteins (e.g., Bcl-2). The ratio of pro-death to pro-survival proteins within a cell generally determines whether the cell undergoes apoptosis or survives.

Bim protein is a pro-death member of the Bcl-2 family. The Bim protein is expressed as at least three protein isoforms of different length (i.e., Bim_{EL}, Bim_{L}, and Bim_{S}) having distinct potencies for promoting cell death, with the Bim_{S} protein isoform being most potent. The Bim protein plays a critical role in mediating cell death in various cell types. Therefore, drugs that modify levels of Bim protein would facilitate treating diseases characterized by abnormally low or abnormally high levels of apoptosis.

The steady-state level of a protein within a cell is generally determined by the rate at which the protein is synthesized relative to the rate at which the protein is degraded (catabolized). The primary mechanism for protein catabolism in cells is the Ubiquitin Proteasome Pathway (UPP). Protein catabolism by the UPP involves two successive steps: (1) covalent attachment of multiple ubiquitin polypeptides to a protein substrate (“ubiquitination”) to produce a ubiquitin-tagged protein; and (2) degradation of the ubiquitin-tagged protein by the 26S proteasome.

FIG. 1 shows a schematic flowchart 50 representing selected aspects of the UPP, including a ubiquitination portion 52 and a degradation portion 54. In the ubiquitination portion, an E3 ubiquitin ligase 56 (also termed an “E3 ligase”) promotes conjugation of a ubiquitin polypeptide 58 to a protein substrate 60. In particular, the ubiquitin polypeptide is transferred to the protein substrate from an E2 ubiquitin-conjugating enzyme 62 (also termed an “E2 enzyme”), which serves as a ubiquitin donor. Before this transfer, in an earlier step not shown here, the E2 enzyme receives ubiquitin polypeptide 58 in an activated form from an E1 ubiquitin-activating enzyme. Then, E3 ligase 56 targets transfer, indicated by an arrow at 64, of ubiquitin polypeptide 58 from E2 enzyme 62 to protein substrate 60. To target this transfer of ubiquitin, the E3 ligase may interact with both the E2 enzyme and the protein substrate at the same time, as shown here, or may interact sequentially such that ubiquitin is received first by the E3 ligase from the E2 enzyme and then is transferred to the protein substrate. In any event, the transfer of a ubiquitin polypeptide to the protein substrate may be performed repeatedly, indicated at 66, to form a ubiquitin-tagged product 68 in which protein substrate 60 is conjugated to a plurality of ubiquitin polypeptides 58. In degradation portion 54 of FIG. 1, ubiquitin-tagged product 68 may be recognized and processed by the 26S proteasome to produce degradation products 70 and released ubiquitin polypeptides 58.

A cell may contain only a few types of E1 enzymes, a larger number of E2 enzymes, and an even greater diversity of E3 ligases. Consistent with this high diversity of E3 ligases employed by the cell, each E3 ligase is thought to play a primary role in substrate identification for degradation in the UPP. However, for most proteins, a corresponding E3 ligase that targets degradation has not yet been identified.

Bim protein has been reported to be ubiquitinated, which suggests that Bim protein is degraded in the Ubiquitin Proteasome Pathway using a Bim-selective E3 ligase. Furthermore, phosphorylation of Bim protein on Ser65 in humans (Ser69 in rat) by p42/p44 MAP kinase (MAPK) has been shown to be an essential step for Bim ubiquitination and proteasomal degradation. As a result, mitogen stimulation of cells, which increases the level of active MAP kinase, may produce increased Bim ubiquitination and degradation, and thus less Bim protein and increased cell survival. However, a Bim-selective E3 ligase that is MAPK-dependent for interaction with Bim protein has not been identified in the prior art, although such an E3 ligase would provide a valuable tool for drug design, clinical diagnostics, and treatment of disease.

### SUMMARY

The present disclosure provides methods, compositions, and cells for drug screening based on interaction between (1) a Bim polypeptide and/or an E2 polypeptide and (2) a TRIM2 polypeptide, which is identical to or corresponds to a Bim-selective E3 ubiquitin ligase. Methods and compositions for treating cancer based on levels of Bim protein, TRIM2 protein, and active MAPK protein are also provided.

### BRIEF DESCRIPTION OF THE DRAWINGS

FIG. 1 is a schematic flowchart of selected aspects of the Ubiquitin Proteasome Pathway.

FIG. 2 is a schematic flowchart of an exemplary model of Bim protein degradation in the Ubiquitin Proteasome Pathway with TRIM2 protein functioning as an E3 ubiquitin ligase for the Bim protein, in accordance with aspects of the present disclosure.

FIG. 3 is a schematic flowchart illustrating an exemplary method of drug screening based on interaction of a Bim polypeptide and a TRIM2 polypeptide, in accordance with aspects of the present disclosure.

FIG. 4 is a schematic flowchart illustrating an exemplary method of drug screening based on interaction of a TRIM2 polypeptide and an E2 ubiquitin-conjugating enzyme polypeptide (i.e., an E2 polypeptide), in accordance with aspects of the present disclosure.

FIG. 5 is a schematic representation of an exemplary method of drug screening based on the use of one or more antibodies to facilitate detecting interaction of Bim and TRIM2 polypeptides, in accordance with aspects of the present disclosure.

FIG. 6 is a schematic representation of an exemplary method of drug screening based on interaction of a Bim polypeptide and a TRIM2 polypeptide each having a distinct luminescent tag, in accordance with aspects of present disclosure.

FIG. 7 is a bar graph presenting exemplary data on Bim protein levels in rat cortical neuronal cultures following preconditioning ischemia, in accordance with aspects of the present disclosure.

FIG. 8 is a bar graph presenting exemplary data on the percentage of dead cells produced in rat cortical neuronal cultures at 24 hours after 120 minutes of harmful ischemia (oxygen-glucose deprivation), with or without 30 minutes of preconditioning ischemia, in accordance with aspects of present disclosure.

FIG. 9 is a series of immunoblots presenting exemplary data on levels of MAPK protein, phospho-MAPK protein (i.e., active MAPK protein), and Bim protein in rat cortical neuronal cultures following preconditioning ischemia, with or without a one hour recovery in the presence of the MAPK kinase inhibitor U0126, in accordance with aspects of present disclosure.

FIG. 10 is an immunoblot presenting exemplary data from interaction of Bim polypeptide with TRIM2 protein provided by a rat brain lysate, in accordance with aspects of the present disclosure.

FIG. 11 is a bar graph presenting exemplary data from interaction of Bim and TRIM2 polypeptides in a cell-free expression system, in accordance with aspects of present disclosure.

FIG. 12 is a bar graph presenting exemplary data on levels of phospho-MAPK protein and Bim protein in a mouse embryonic fibroblast cell line, in accordance with aspects of present disclosure.

FIG. 13 is a bar graph presenting exemplary data on interaction of a Bim polypeptide with TRIM2 protein from a mouse embryonic fibroblast cell line, in accordance with aspects of present disclosure.

FIG. 14 is a bar graph presenting exemplary data on interaction of a Bim polypeptide with TRIM2 protein under conditions that promote Bim ubiquitination in rat cortical neuronal cultures, in accordance with aspects of present disclosure.

FIG. 15 is a bar graph presenting exemplary data on ubiquitination of Bim protein in neuronal cell lysates with and without TRIM2 depletion using an anti-TRIM2 antibody, in accordance with aspects of present disclosure.

FIG. 16 is a graph presenting exemplary data on TRIM2 and Bim protein levels in neurons, with and without interference of TRIM2 expression using a short hairpin RNA, in accordance with aspects of present disclosure.

FIG. 17 is a graph presenting exemplary data on Bim and TRIM2 protein expression ratios in established breast cancer cell lines, in accordance with aspects of present disclosure.

FIG. 18 is a pair of immunoblots presenting exemplary data on expression of TRIM2 and Bim proteins in breast tumor samples, in accordance with aspects of present disclosure.

FIG. 19 is a graph of the expression data of FIG. 18.

FIG. 20 is a bar graph presenting exemplary data on interaction of TRIM2 with various E2 ubiquitin-conjugating enzymes, in accordance with aspects of present disclosure.

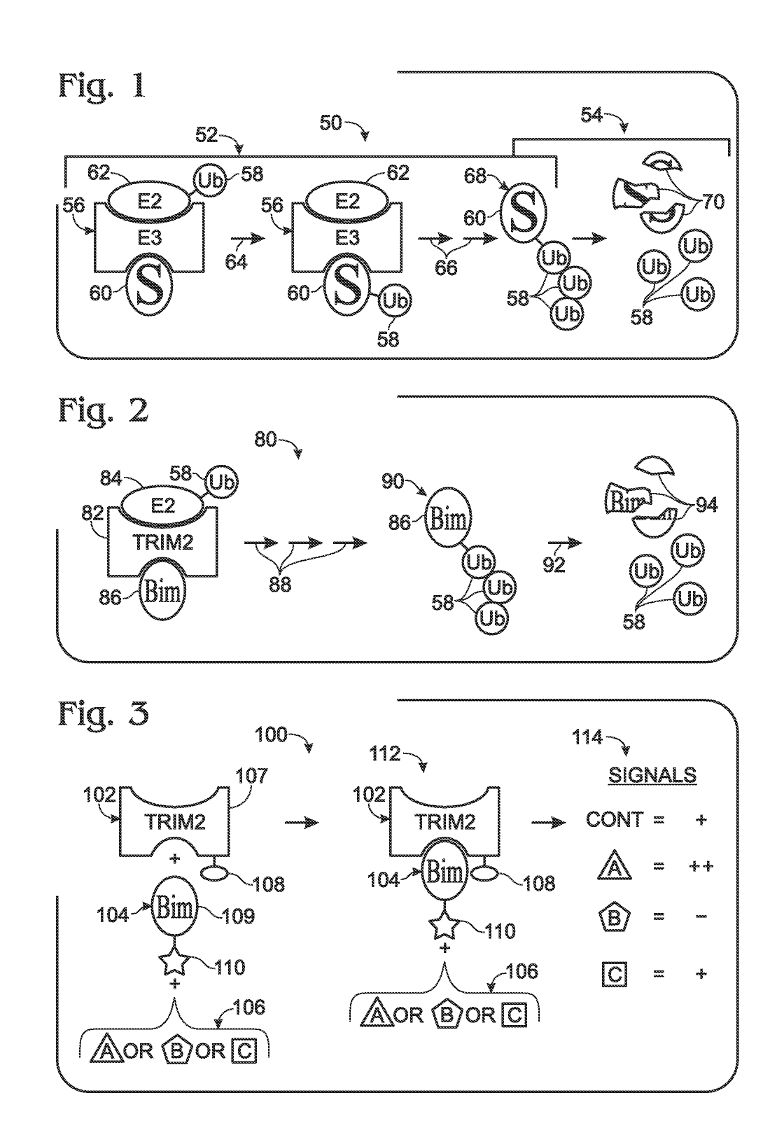

### DETAILED DESCRIPTION

The present disclosure provides methods, compositions, and cells for drug screening based on interaction between (1) a Bim polypeptide and/or an E2 polypeptide and (2) a TRIM2 polypeptide, which is identical to or corresponds to a Bim-selective E3 ubiquitin ligase. Methods and compositions for treating cancer based on levels of Bim protein, TRIM2 protein, and active MAPK protein are also provided.

The method for drug screening may be used to develop new drugs for enhancing or inhibiting apoptosis. An assay mixture may be formed. The assay mixture may include a Bim polypeptide and a TRIM2 polypeptide (or an E2 polypeptide and a TRIM2 polypeptide) that interact with each other. In some embodiments, one or both of the Bim and TRIM2 (or E2 and TRIM2) polypeptides may be tagged, such as with an epitope tag, a luminescent tag, an enzyme tag, an affinity tag, or any combination thereof, among others. For example, the Bim and TRIM2 (or E2 and TRIM2) polypeptides may be tagged with distinct luminescent tags, such as having respective Bim and TRIM2 (or E2 and TRIM2) portions conjugated to distinct fluorescent polypeptides. The assay mixture also may include one or more test compounds. Furthermore, the assay mixture also or alternatively may include a MAPK (since interaction between Bim and TRIM2 polypeptides may be increased by and/or dependent on MAPK activity), a cell extract, biological cells, or a combination thereof, among others. The biological cells may express one or both of the Bim and TRIM2 (or E2 and TRIM2) polypeptides, for example, both polypeptides may be expressed by the same cells or by respective distinct cells. Interaction of the Bim and TRIM2 (or E2 and TRIM2) polypeptides with each other in the assay mixture may be detected. Whether the one or more test compounds affect the interaction of the Bim and TRIM2 (or E2 and TRIM2) polypeptides may be determined. At least one test compound then may be selected as a candidate drug for treatment of a disease characterized by an abnormal amount of cell death/apoptosis. The selection may be based at least in part on a determination that the at least one test compound affects interaction of the Bim and TRIM2 (or E2 and TRIM2) polypeptides.

The present disclosure also provides a composition for drug screening to develop new drugs for enhancing or inhibiting apoptosis. The composition may include engineered Bim and TRIM2 (or E2 and TRIM2) polypeptides that interact with one another. The composition further may include biological cells that express the engineered polypeptides.

The present disclosure further provides method of treating cancer, such as breast cancer. One or more samples from a cancer patient may be tested for levels of TRIM2 protein (or RNA), Bim protein (or RNA), and active MAPK protein (or MAPK RNA). The cancer patient may be treated with at least one drug that inhibits degradation of Bim protein if the level of Bim protein (or RNA) is below a first threshold, the level of TRIM2 protein (or RNA) is above a second threshold, the level of active MAPK (or MAPK RNA) is above a third threshold, or any combination thereof. Exemplary drugs that may be used for treatment include an inhibitor of proteasome activity or MAPK activity (e.g., via upstream inhibition of a MAPK kinase), among others.

FIG. 2 shows a schematic flowchart 80 of a model of Bim protein degradation in the Ubiquitin Proteasome Pathway. A TRIM2 protein 82 may function as a Bim-selective E3 ubiquitin ligase that promotes transfer of ubiquitin 58 from an E2 ubiquitin-conjugating enzyme 84 (an E2 enzyme) to a Bim protein 86. Accordingly, ubiquitin 58 may be transferred repeatedly, indicated by arrows at 88, from E2 enzyme 84 to Bim protein 86 to form a polyubiquitinated Bim protein 90. The polyubiquitinated Bim protein may be degraded by the 26S proteasome, indicated by the arrow at 92, to form Bim degradation products 94 and released ubiquitin polypeptides 58.

The mechanistic details presented in flowchart 80 are illustrative only and are not intended to provide a theory of operation that limits the scope of the claimed invention. For example, interaction of TRIM2 protein with Bim protein and/or E2 enzyme may involve other polypeptides not shown here and thus may or may not be via direct contact. Moreover, interaction of TRIM2 protein with Bim protein may be increased or may at least substantially require phosphorylation of the Bim protein, such as by MAPK. In addition, TRIM2 protein may not interact with E2 enzyme and Bim protein at the same time. Also, the interactions may or may not involve a dimer or higher order complex of E2 enzyme, TRIM2 protein, and/or Bim protein. Furthermore, Bim protein may be degraded as a monoubiquitinated form.

The present disclosure identifies the TRIM2 protein as an E3 ligase for Bim protein based on several lines of evidence (e.g., see Section VI). (1) TRIM2 protein was identified by its interaction with Bim protein in a complex mixture of proteins. (2) TRIM2 protein has a RING finger domain, which is a structural domain commonly found in E3 ubiquitin ligases. (3) The present disclosure presents data showing that Bim and TRIM2 polypeptides interact in several different assay systems, including a cell-free expression system and a cell lysate. (4) The present disclosure presents data showing that expression of Bim and TRIM2 proteins is inversely correlated in various cancer lines and tumor samples and also when TRIM2 protein expression is reduced by RNA interference. (5) The present disclosure presents data showing that interaction of Bim and TRIM2 polypeptides is inhibited by treatments that promote apoptosis, such as ischemia and upstream inhibition of MAPK activity in neuronal cultures. (6) The present disclosure presents data showing that a TRIM2 polypeptide interacts with several distinct E2 ubiquitin-conjugating enzymes.

The following sections describe further aspects of the present disclosure, including, among others, (I) interaction assays, (II) Bim, TRIM2, and E2 polypeptides, (III) test compounds, (IV) selecting a test compound as a candidate drug, (V) treating cancer based on testing for Bim and TRIM2 proteins, and (VI) examples.

### I. Interaction Assays

FIG. 3 shows a schematic flowchart 100 illustrating an exemplary method of drug screening based on an interaction assay involving Bim and TRIM2 polypeptides. The assay may be performed using (1) a TRIM2 polypeptide 102, (2) a Bim polypeptide 104, and (3) one or more test compounds 106 (indicated here as A-C). One or both of TRIM2 polypeptide 102 and Bim polypeptide 104 may be tagged. For example, the TRIM2 polypeptide may include a TRIM2 portion 107 attached to a first tag 108. Alternatively, or in addition, the Bim polypeptide may include a Bim portion 109 attached to a second tag 110. Each tag may (or may not) be a polypeptide and may have any suitable size relative to its respective Bim or TRIM2 portion, such as being smaller or larger than the respective Bim or TRIM2 portion.

At least one assay mixture 112 including TRIM2 and Bim polypeptides 102, 104 and test compounds 106 may be formed. For example, each test compound (A-C) may be disposed in a respective, separate assay mixture with the Bim and TRIM2 polypeptides or two or more test compounds may be disposed in the same assay mixture with the Bim and TRIM2 polypeptides. In some embodiments, a plurality of assay mixtures may be formed in respective wells of a microplate (e.g., a microtiter dish). Exemplary microplates may include 96, 384, or 1536 wells, among others. Furthermore, no test compound may be disposed in at least one assay mixture to provide a control.

The assay mixture may be formed by combining any suitable materials in any suitable order. The assay mixture may include cells that express one or both of the Bim and TRIM2 polypeptides, a cell extract containing one or both of the Bim and TRIM2 polypeptides, a kinase (e.g., a MAPK), a MAPK kinase (e.g., MEK1 and/or MEK2) inhibitor (e.g., PD98059, U0126, SL 327, etc.), or any combination thereof. At least one component of the assay mixture (e.g., TRIM2 polypeptide 102 or Bim polypeptide 104) may be connected to a solid support (e.g., a membrane, a vessel wall, a bead, or the like).

Interaction of Bim and TRIM2 polypeptides 102, 104 in the assay mixtures may be detected as signals 114. For example, here, detected signals are listed for a control (“CONT”; no test compound) and test compounds A-C individually. The signals may be detected directly from the assay mixtures in a homogeneous assay, without separating bound and free polypeptides, such as in a homogeneous optical assay, or may be detected after performing at least one separation procedure on each assay mixture in a heterogeneous assay. The detected signals may be analyzed to determine whether the test compounds affect the interaction of Bim and TRIM2 polypeptides 102, 104. Analysis of the detected signals may include comparing a signal to a threshold value and/or to a control value, among others. For example, here, comparison of the detected signals shows that test compounds A, B, and C respectively increase, decrease, and have no effect on the interaction of Bim and TRIM2 polypeptides. In some examples, at least one test compound may be selected as a drug candidate for regulating apoptosis (i.e., increasing or decreasing apoptosis) based at least in part on a determination that the test compound affects the Bim-TRIM2 interaction.

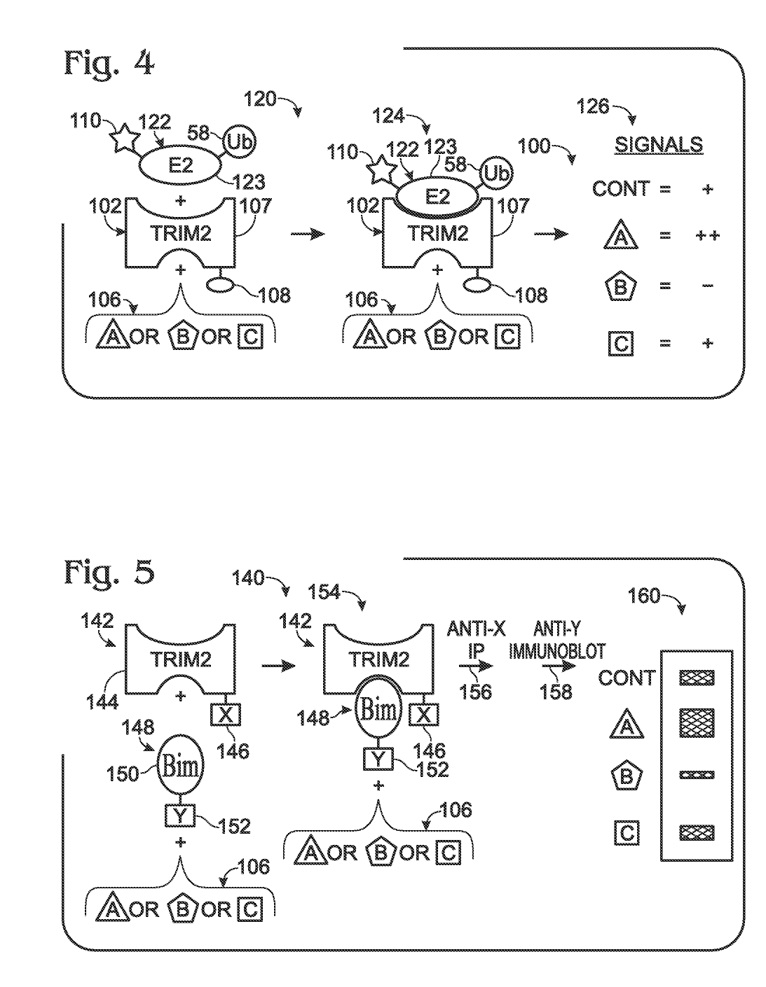

FIG. 4 shows a schematic flowchart 120 illustrating an exemplary method of drug screening based on an assay for interaction of a TRIM2 polypeptide and an E2 ubiquitin-conjugating enzyme polypeptide (“E2 polypeptide”). The assay may be performed using (1) TRIM2 polypeptide 102, (2) an E2 polypeptide 122 that corresponds or is identical to a particular E2 enzyme, and (3) one or more test compounds 106. One or both of TRIM2 polypeptide 102 and E2 polypeptide 122 may be tagged, such as including respective first and second tags 108, 110. Accordingly, the E2 polypeptide may include an E2 portion 123 attached to tag 110, and further may be conjugated to ubiquitin polypeptide 58.

At least one assay mixture 124 including TRIM2 and E2 polypeptides 102, 122 and one or more of test compounds 106 may be formed, and signals may be detected, indicated at 126. The assay mixture may include any of the components described above for Bim-TRIM interaction assays, and optionally may include a polypeptide corresponding or identical to Bim protein, an E1 ubiquitin-activating enzyme, ubiquitin, ATP, a MAPK (particularly an active MAPK, such as active ERK1/2), or any combination thereof. At least one component of the assay mixture (e.g., TRIM2 polypeptide 102, E2 polypeptide 122, or a Bim polypeptide, among others) may be connected to a solid support.

FIG. 5 shows a schematic flowchart 140 illustrating an exemplary method of drug screening based on the use of one or more antibodies to facilitate detecting interaction of Bim and TRIM2 polypeptides. The assay may be performed using a TRIM2 polypeptide 142 that includes a TRIM2 portion 144 conjugated to a first epitope tag 146 (“X”), a Bim polypeptide 148 that includes a Bim portion 150 conjugated to a second epitope tag 152 (“Y”), and one or more test compounds 106 (e.g., A-C). In some embodiments, one or both of the epitope tags may be omitted. An exemplary first (or second) epitope tag 146 that may be suitable is a c-myc tag recognized by a monoclonal antibody (9E10) produced by immunizing mice with a synthetic peptide containing a c-myc epitope. An exemplary second (or first) epitope tag 152 that may be suitable is an HA tag that is recognized by a monoclonal antibody (6E2) produced by immunizing mice with a synthetic peptide containing an influenza hemagglutinin epitope.

The method may include any combination of the following exemplary procedures. At least one assay mixture 154 may be formed that includes TRIM2 polypeptide 142, Bim polypeptide 148, and one or more test compounds 106. The assay mixture may use any suitable buffer, salt, detergent, chelator, etc. An exemplary Tris buffer cocktail that may have a suitable composition for interaction assays includes 150 mM NaCl, 50 mM Tris-HCl (pH 8.0), 1.0% NP-40, 1 mM EDTA, and optionally may include protease inhibitors (aprotinin 1 μg/mL, leupeptin 2 μg/mL, pepstatin 1 μg/mL, and PMSF (10 μg/mL). The Tris buffer cocktail may be used to make a cell extract.

The assay mixture may be contacted with a first antibody, indicated at 156, that specifically binds to only one of polypeptides 142, 148. For example, the assay mixture may be contacted with an antibody against first epitope tag 146 or second epitope tag 152. Here, the first antibody is an anti-X antibody that recognizes the first epitope tag. Alternatively, the assay mixture may be contacted with an anti-TRIM2 antibody or an anti-Bim antibody that recognizes TRIM2 protein or Bim protein, respectively. In some embodiments, contacting the assay mixture with an antibody may bind one of polypeptides 142, 148 (“a first polypeptide”) to a solid support, such as a bead, a membrane, or a vessel wall, among others. In some embodiments, after contacting the assay mixture with the first antibody, the first antibody and its bound first polypeptide may be separated from the assay mixture, such as by performing immunoprecipitation and/or one or more washing steps.

Material separated with the first antibody then may be analyzed for the presence of the other polypeptide (“the second polypeptide”), resulting from co-immunoprecipitation with (and thus interaction with) the first polypeptide, by any suitable mechanism, such as by contact with a second antibody that specifically binds the second polypeptide. For example, the separated material may be resolved by gel electrophoresis followed by immunoblotting with the second antibody, indicated at 158, to provide signals 160 indicative of the amount of interaction between TRIM2 and Bim polypeptides in the presence of each test compound (A-C) relative to control (CONT). Here, the second antibody is an anti-Y antibody that recognizes the second epitope tag. In other embodiments, an antibody recognizing the TRIM2 polypeptide may be used for immunoprecipitation and an antibody recognizing the Bim polypeptide may be used for immunoblotting. In other embodiments, one or both of the Bim and TRIM2 polypeptides may be recognized using an antibody directed against Bim or TRIM2 protein.

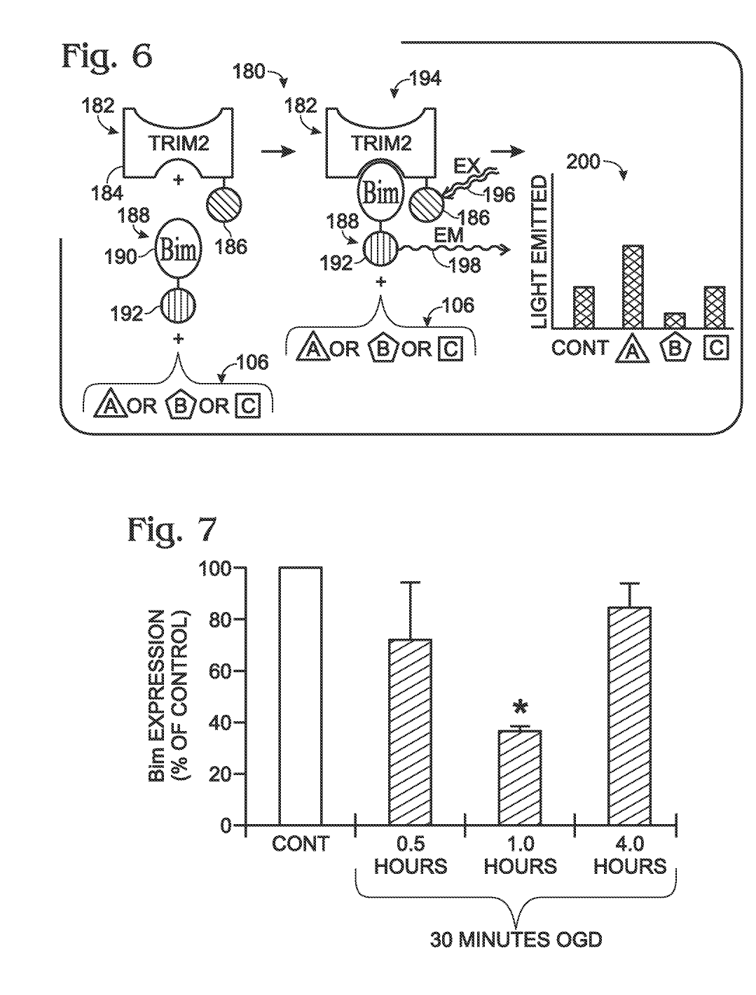

FIG. 6 shows a schematic flowchart 180 illustrating an exemplary method of drug screening based on the use of Bim and TRIM2 polypeptides each having a distinct luminescent tag. The method may be performed using a TRIM2 polypeptide 182 that includes a TRIM2 portion 184 conjugated to a first luminescent tag 186, a Bim polypeptide 188 that includes a Bim portion 190 conjugated to a second luminescent tag 192, and one or more test compounds 106 (e.g., A-C). At least one assay mixture 194 may be formed including TRIM2 polypeptide 182, Bim polypeptide 188, and one or more of test compounds 106.

First and second luminescent tags, which also may be termed fluorescent tags, may be a transfer pair that promotes energy transfer from the first to the second luminescent tag. Energy transfer is the transfer of luminescence energy from a donor luminophore to an acceptor without emission by the donor. In energy transfer assays, a donor luminophore is excited from a ground state into an excited state by absorption of a photon. If the donor luminophore is sufficiently close to an acceptor, excited-state energy may be transferred from the donor to the acceptor, causing donor luminescence (and donor lifetime) to decrease and acceptor luminescence to increase (if the acceptor is luminescent). The efficiency of this transfer is very sensitive to the separation distance, R, between donor and acceptor, decaying as 1/R⁶. Energy transfer assays use energy transfer to monitor the proximity of donor and acceptor. Accordingly, when donor and acceptor luminescent tags are conjugated to a Bim portion and a TRIM2 portion, the energy transfer will be more efficient when the Bim and TRIM2 polypeptides are interacting with one another. In exemplary embodiments, the first and second luminescent tags may be distinct fluorescent polypeptides, such as distinct green fluorescent protein (GFP) polypeptides (e.g., cyan fluorescent protein, yellow fluorescent protein, red fluorescent protein, etc.) which may have distinct excitation and/or emission spectra. The first and second luminescent tags may be conjugated to the Bim and TRIM2 portions of their respective polypeptides at the amino terminus or the carboxy terminus of the Bim or TRIM2 portion, or may be disposed intermediate the amino and carboxy termini. In some examples, the first and second luminescent tags may have substantially nonoverlapping excitation spectra and/or emission spectra.

The spectral properties of the luminescent tags may be characterized by excitation spectrum, emission spectrum, and/or Stokes' shift, among others. An excitation spectrum is the dependence of emission intensity upon the excitation wavelength, measured at a single constant emission wavelength. An emission spectrum is the wavelength distribution of the emission, measured after excitation with a single constant excitation wavelength. A Stokes' shift is the difference in wavelengths between the maximum of the emission spectrum and the maximum of the absorption spectrum. In some examples, the emission spectrum of the first luminescent tag may overlap the excitation spectrum of the second luminescent tag, such that energy emitted from excitation of the first luminescent tag may function as excitation energy for the second luminescent tag. Accordingly, by appropriate selection of excitation light, the first luminescent tag may be selectively excited directly and the second luminescent tag may be selectively excited indirectly.

In the present illustration, due to the presence of luminescent tags 186, 192, interaction of TRIM2 polypeptide 182 and Bim polypeptide 188 may be detected optically. For example, first luminescent tag 186 may be selectively excited using excitation light 196. Excitation energy produced by the excitation light may be transferred to second luminescent tag 192 by energy transfer. The transferred energy may excite the second luminescent tag, which may result in production of emitted light 198 from the second luminescent tag.

Optically detected signals corresponding to the amount of light emitted are indicated at 200. In the present illustration, relative to control (CONT), test compound A causes more light to be emitted (indicating more Bim-TRIM2 interaction), test compound B causes less light to be emitted (indicating less Bim-TRIM2 interaction), and test compound C has no effect on the amount of emitted light (indicating no effect on Bim-TRIM2 interaction). In other embodiments, second luminescent tag 192 may quench light emission when it is in proximity to the first luminescent tag, such that the amount of emitted light decreases with more Bim-TRIM2 interaction. Alternatively, or in addition, second luminescent tag 192 may change the fluorescence lifetime of the first luminescent tag. Accordingly, optically detected signals may measure fluorescence intensity or fluorescence lifetime, among others.

In exemplary embodiments, interaction of Bim and TRIM2 polypeptides may be detected by fluorescence resonance energy transfer (FRET) using a system generally described for other proteins in the following reference, which is incorporated herein by reference: Nguyen, A. W. and P. S. Daugherty, Evolutionary optimization of fluorescent proteins for intracellular FRET. Nat. Biotechnol., 2005. 23(3): pp. 355-360. For example, a Bim (or TRIM2) polypeptide may be tagged with YPet (a green fluorescent protein variant that is yellow) and a TRIM2 (or Bim) polypeptide may be tagged with CyPet (a green fluorescent protein variant that is blue). Each polypeptide may be tagged with YPet or CyPet by constructing a fusion polynucleotide coding sequence for each respective fusion polypeptide. For example, the polynucleotide coding sequence for YPet may be fused, in-frame, at a position corresponding to the amino terminus of Bim in a Bim polynucleotide coding sequence, to produce a YPet-Bim fusion coding sequence. In addition, the polynucleotide coding sequence for CyPet may be fused, in-frame, at a position corresponding to the amino terminus of TRIM2 in a TRIM2 polynucleotide coding sequence, to produce a CyPet-TRIM2 fusion coding sequence. YPet-Bim and CyPet-TRIM2 fusion polypeptides may be expressed from the corresponding fusion coding sequences. The fusion polypeptides may be expressed from expression vectors introduced into the same cells, to provide an optical assay performed in cells. Alternatively, or in addition, the fusion polypeptides may be isolated from the cells or expressed in a cell-free expression system, to provide an optical assay performed outside of cells.

Fluorescence polarization alternatively may provide a measure of interaction. In this case, only one of the Bim and TRIM2 polypeptides may include a luminescent tag. Fluorescence polarization signals may be detected in assay mixtures including test compounds, and the effect of the test compounds on Bim-TRIM2 interaction may be determined based on the detected signals. In particular, the extent of polarization measured from the luminescent tag may be inversely related to the amount of interaction between the Bim and TRIM2 polypeptides, because polarization will increase when the luminescent tag is part of a larger complex produced by Bim-TRIM2 interaction.

In other optical assays for interaction, the assay may detect total internal reflection luminescence (e.g., TIRF), luminescence correlation spectroscopy (e.g., FCS), and/or luminescence recovery after photobleaching (e.g., FRAP or FPR), among others.

### II. Bim, TRIM2, and E2 Polypeptides

The methods, compositions, and cells disclosed herein may use and/or include at least one Bim polypeptide, at least one TRIM2 polypeptide, at least one E2 polypeptide, or any combination thereof. A “polypeptide,” as used herein, is a covalently linked chain of amino acids. The chain may have any suitable number of amino acids, such as at least 10, 20, 50, or more amino acids, among others.

A Bim polypeptide, a TRIM2 polypeptide, and an E2 polypeptide, as used herein, are polypeptides that correspond to, or are identical to, a respective full-length, native Bim protein, TRIM2 protein, and E2 ubiquitin-conjugating enzyme protein. A polypeptide that corresponds to a protein has a sequence that is based, at least in part, on that protein, such that the polypeptide is detectably related to that protein using an alignment algorithm or by visual inspection. For example, a polypeptide that corresponds to a protein may have a subsequence of at least 10 amino acids that has at least 90% or 100% amino acid identity to a subsequence of that protein, and/or the polypeptide may have a subsequence of at least 25, 50, or 100 amino acids that has at least 80%, 90%, 95% or 100% amino acid identity to a subsequence of that protein, when the polypeptide and protein are aligned for maximum correspondence. A Bim, TRIM2, or E2 polypeptide thus may differ from a respective full-length, native Bim, TRIM2, or E2 protein by at least one amino acid substitution, one or more deletions of one or more amino acids, one or more additions of one or more amino acids, or a combination thereof. A Bim, TRIM2, or E2 polypeptide may be an engineered polypeptide, which means that the polypeptide has been designed by man to differ in at least one position from a respective Bim, TRIM2, or E2 protein.

A Bim, TRIM2, or E2 polypeptide may include or be only a fragment of a respective Bim, TRIM2, or E2 protein. The fragment may include or be only an amino terminal fragment, a carboxy terminal fragment, or an internal fragment of the respective protein. The fragment may represent any suitable portion of the respective protein, such as less or more than one-fourth or one-half, among others. In some cases, a Bim, TRIM2, or E2 polypeptide may be a fusion polypeptide that includes a foreign polypeptide not found in a respective Bim, TRIM2, or E2 protein. The foreign polypeptide may function as a tag.

A Bim, TRIM2, or E2 polypeptide may correspond most closely to, or be identical to, a respective Bim, TRIM2, and E2 protein from any suitable species. The species may, for example, be a vertebrate, such as a mammal. Exemplary mammals that may be suitable include primates (e.g., humans), rodents, canines, felines, etc.

A Bim polypeptide may correspond most closely to, or be identical to, any suitable isoform of Bim. For example, the Bim polypeptide may correspond most closely to, or be identical to, Bim_{EL}, Bim_{L}, or Bim_{S}.

A TRIM2 polypeptide may correspond to, or be identical to, a TRIM2 protein. TRIM2, which stands for tripartite motif-containing 2, may also be known as RNF86 and KIAA0517. The TRIM2 protein may include a tripartite motif, namely, a RING domain, at least one B-box, and a coiled-coil region (collectively termed RBCC). Accordingly, the TRIM2 polypeptide may have any suitable combination of these motifs.

An E2 polypeptide may correspond most closely to, or be identical to, any suitable E2 ubiquitin-conjugating enzyme. Exemplary E2 enzymes that may be suitable include Ubc 1, 2, 3, 4, 5, 6, 7, 8, 10, or 13, among others.

The present disclosure also provides cells that express one or more engineered polypeptides. For example, the cells may express a Bim, a TRIM2, an E2, Bim and TRIM2, or E2 and TRIM2 engineered polypeptides. In some embodiments, the cells may express at least one engineered polypeptide (i.e., Bim, TRIM2, or E2) and may be engineered to express at least one other polypeptide (i.e., Bim, TRIM2, E2, or MAPK). The term “engineered to express,” as used herein, means that cells are designed to produce the at least one other polypeptide using a polynucleotide template introduced into the cells by human action.

Any of the Bim, TRIM2, and E2 polypeptides disclosed herein may include a tag that is attached covalently or noncovalently to a respective Bim, TRIM2, or E2 portion. The term “conjugated” is intended to mean covalently attached. A tag may be conjugated translationally or postranslationally to a Bim, TRIM2, or E2 portion. In exemplary embodiments, a Bim, TRIM2, or E2 polypeptide is a fusion polypeptide encoded by a recombinant coding sequence, which may be expressed in cells or in a cell-free system, among others. The tag may, for example, be an enzyme tag, that is, a tag having a measurable enzyme activity. Exemplary enzyme tags may include alkaline phosphatase, beta-galactosidase, horseradish peroxidase, etc. Alternatively, or in addition, the tag may be an affinity tag, that is, a tag that is bound by a specific binding partner, such as an antibody, biotin, a metal ion, an enzyme substrate, or the like. Exemplary affinity tags may include beta-galactosidase, (His)₆, glutathione S-transferase, maltose binding protein, avidin/streptavidin, or the like.

### III. Test Compounds

The methods and compositions disclosed herein may use or include one or more test compounds. A “test compound,” as used herein, is any chemical substance that is evaluated for activity. The test compounds may be molecules of any suitable size, such as small molecules of less than about 10 kDa. The test compounds may be evaluated individually or as one or more groups of two or more test compounds each. In some examples, the test compounds may form a set of related molecules, such as a set of polynucleotides, polypeptides, sugars, lipids, or the like. In some examples, the test compounds may be a set of polypeptides corresponding to a Bim protein, such as corresponding to distinct fragments of the Bim protein.

### IV. Selecting a Test Compound as a Candidate Drug

The methods disclosed herein may involve selection of a test compound as a candidate drug based, at least in part, on an ability of the test compound to affect interaction of Bim and TRIM2 polypeptides or E2 and TRIM2 polypeptides. A test compound may be selected as a candidate drug for treating any suitable medical condition and based on any suitable effect on the interaction. For example, a test compound may be selected as a candidate drug for treating cancer based at least in part on the ability of the test compound to decrease interaction of Bim and TRIM2 polypeptides or E2 and TRIM2 polypeptides relative to control. Accordingly, the test compound may be utilized in the therapy of cancer and similar diseases where enhanced cell survival or cell resilience to chemotherapeutic agents is part of the pathology of the disease. Additional therapeutic implications may be identified in sepsis, which involves aberrant cell death signaling. Alternatively, a test compound may be selected as a candidate drug to provide a neuroprotective effect for treating a neurological disease where deregulated apoptosis (e.g., acute or prolonged periods of neurodegeneration) is a feature (e.g., stroke, Alzheimer's disease, epilepsy, Parkinson's disease, etc.) based at least in part on an ability of the test compound to increase interaction of Bim and TRIM2 polypeptides or E2 and TRIM2 polypeptides relative to control.

### V. Treating Cancer Based on Testing for Bim and TRIM2 Proteins

The present disclosure provides a method of treating a patient for cancer based on analysis of the status of a Bim ubiquitination pathway in the patient. In particular, the patient may be treated if the level of Bim is low and if the Bim ubiquitination pathway is predicted to be active and thus is a potential cause of the low Bim level. By decreasing the level of Bim ubiquitination and degradation, apoptosis is promoted, thereby killing cancer cells.

One or more samples may be collected from the patient. The samples may have any suitable origin in the patient, such as a tumor, blood, urine, a fluid aspirate, or the like.

The samples may be tested for a level of a TRIM2 protein (or RNA), a Bim protein (or RNA), an active MAPK protein (or MAPK RNA), or any combination thereof. For example, the same sample may be tested for levels of two or more of the proteins (or RNAs) or at least two samples may be tested for respective distinct proteins. Testing may be performed by any suitable procedure for each protein, such as Western blot, dot blot, enzyme-linked immunosorbent assay (ELISA), competitive immunoprecipitation, mass spectrometry, or the like.

The patient may be treated with at least one drug that inhibits degradation of Bim protein if the tested levels meet predetermined criteria. In particular, the patient may be treated with the drug if selected components of the Bim ubiquitination pathway are present above or below particular thresholds. For example, the patient may be treated with the drug if the tested sample indicates that the level of Bim protein (or RNA) may be limiting for apoptosis, that is, the level of Bim protein is below a first threshold. In addition, the patient may be treated with the drug if the tested sample indicates that the low level of Bim protein may be caused in part by a relatively high level of TRIM2, that is, the level of TRIM2 protein (or RNA) is above a second threshold. Furthermore, the patient may be treated with the drug if the tested sample indicates that the low level of Bim protein may be caused in part by Bim-TRIM2 interaction promoted by Bim phosphorylation via MAPK, that is, the sample has a relatively high level of active MAPK protein (i.e., phosphorylated MAPK). In particular, the level of active MAPK protein (or MAPK RNA) is above a third threshold.

Any suitable drug may be used to inhibit Bim degradation in the patient. Exemplary drugs include a proteasome inhibitor (e.g., bortezomib, MG-132, MG-115, PSI, epoxomicin, lactacystin, etc.), a MAPK kinase (MEK) inhibitor (e.g., PD98059, U0126, SL 327, etc.), or a combination thereof.

### VI. Examples

The following examples describe selected aspects and embodiments of the present disclosure, including (1) analysis of Bim protein in neuronal cultures exposed to ischemic conditions, (2) identification and confirmation of TRIM2 protein as an E3 ubiquitin ligase for Bim, (3) analysis of Bim and TRIM2 protein expression in cancer cell lines and tumors, and (4) data related to interaction of TRIM2 polypeptide with E2 ubiquitin-conjugating enzymes. These examples and the various features and aspects thereof are included for illustration and are not intended to define or limit the entire scope of the present disclosure. The various features and aspects of the following examples may be combined with one another or introduced into any of the other methods or compositions of the present disclosure in any suitable combination.

### Example 1

### Analysis of Bim, MAPK, and Apoptosis in Neuronal Cultures

This example describes experiments performed with cultured neurons to define the relationship between Bim degradation, short-term ischemic tolerance, and MAPK activity; see FIGS. 7-9.

Tolerance is the phenomenon whereby previous exposure to a subtoxic insult conditions a cell or organism against a more severe toxic challenge. Tolerance appears to be a conserved endogenous phenomenon and has been described in multiple organisms from simple cells to more complex systems. Ischemic tolerance has been observed in the brain; a brief preconditioning ischemic insult results in a reduced activation of cell death pathways that occur following a harmful ischemic event.

Two types of ischemic tolerance, delayed and rapid, have been reported for the brain. Delayed ischemic tolerance develops over 1-3 days in vivo or 24 h in vitro, is mediated by a gene-based mechanism, and requires new protein synthesis. In contrast, rapid ischemic tolerance is protein synthesis-independent and occurs within one hour of a preconditioning ischemia.

The experiments described in this example investigate the degradation of the pro-apoptotic Bcl-2 family member Bim as a mechanism of rapid ischemic tolerance. Bim protein levels were observed to be reduced one hour following preconditioning and occurred concurrent with an increase in Bim ubiquitination. Ubiquitinated proteins are degraded by the proteasome, and inhibition of the proteasome with MG-132 prevented Bim degradation and blocked rapid ischemic tolerance. Inhibition of p42/p44 mitogen-activated protein kinase (MAPK) activation by U0126 reduced Bim ubiquitination and Bim degradation and blocked rapid ischemic tolerance.

FIG. 7 shows a bar graph presenting exemplary data on Bim protein levels in rat cortical neuronal cultures following preconditioning ischemia. Neurons were exposed to ischemic conditions, namely, oxygen and glucose deprivation (OGD), by washing the cells with phosphate-buffered saline (NaCl (1.37 mM), KCl (2.7 mM), Na₂HPO₄ (10 mM), KH₂PO₄ (1.7 mM), pH 7.4) supplemented with 0.5 mM CaCl₂, 1.0 mM MgCl₂ and placing the cells (in culture dishes) in an anaerobic chamber (85% N₂, 5% H₂, 10% CO₂; 35° C.) for 30 minutes (preconditioning ischemia) or 120 minutes (harmful ischemia).

Bim protein levels were determined by immunoblotting, and then the blots were re-probed with α-tubulin to control for loading. Data were analyzed by one-way ANOVA with Bonferroni's post-hoc test. *, p>0.05, n=4. Bim protein levels were decreased in cortical cultures one hour following 30 min of OGD (preconditioning) but recovered to normal levels at four hours. In contrast, the expression of the pro-apoptotic cell death proteins Bid and Bax did not change (not shown).

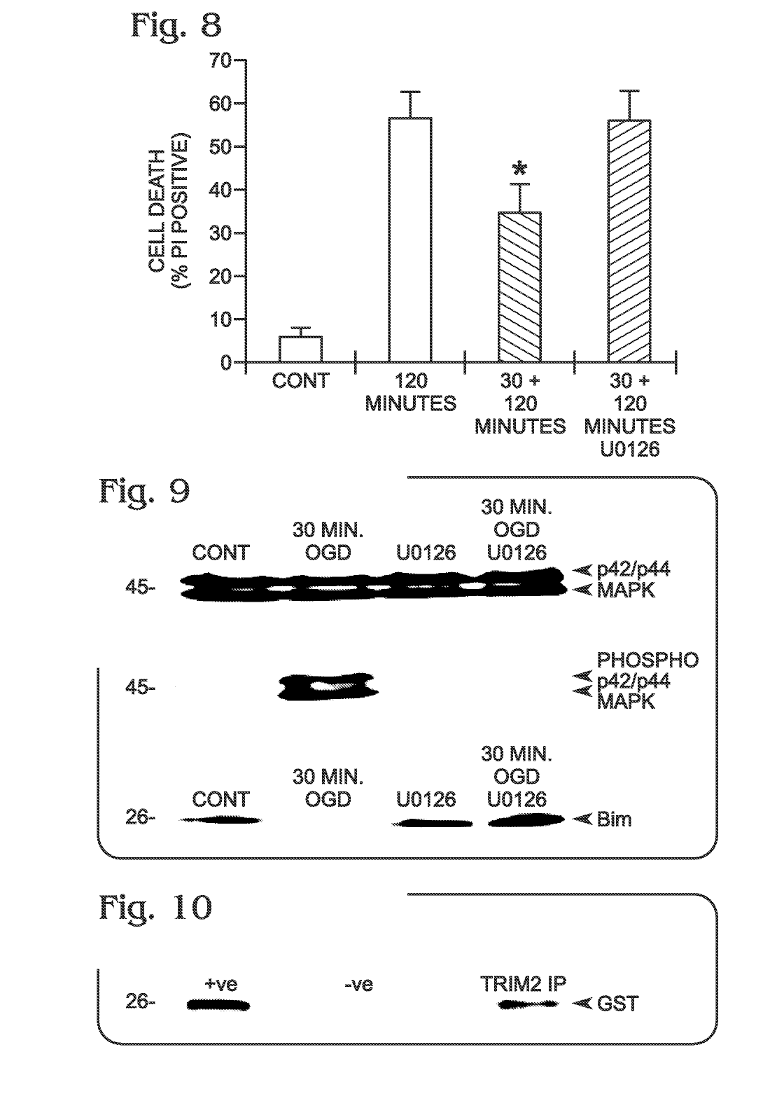

FIG. 8 shows a bar graph presenting exemplary data on the percentage of dead cells produced in rat cortical neuronal cultures at 24 hours after 120 minutes of harmful ischemia, with (hatched bars) or without (open bars) 30 minutes of preconditioning ischemia, and with or without a one-hour recovery with U0126. Cell death was assessed 24 hours following 120 minutes of harmful ischemia using propidium iodide staining. Data shown are mean±S.E. (n=8). Data were analyzed by one-way ANOVA, with Bonferroni's post-hoc test. *, p<0.05.

The phosphorylation and resultant ubiquitination of Bim may be regulated by p42/p44 MAPK; hence the effect of inhibition of p42/p44 MAPK on rapid ischemic tolerance and Bim degradation was determined. Cells were incubated for one hour following 30 minutes of preconditioning OGD with either PD98059 or U0126 (both 10 μM), which are compounds that prevent p42/p44 MAPK activation by inhibiting an upstream regulatory MAPK kinase (MEK). (The upstream regulatory protein kinase MEK may activate p42/p44 MAPK via the phosphorylation of Thr202/Tyr204 residues.) Both U0126 and PD98059 (not shown) blocked the neuroprotective effect of preconditioning.

FIG. 9 shows a series of immunoblots presenting exemplary data on levels of MAPK, phospho-MAPK, and Bim protein in rat cortical neuronal cultures following preconditioning ischemia for 30 minutes, with or without a one-hour recovery in the presence of the MAPK kinase inhibitor U0126. In the top and middle panels of the figure, phosphorylation of p42/p44 MAPK was determined by immunoblot, and the blots were re-probed for total p42/p44 MAPK expression. In the bottom panel of the figure, cells were subjected to preconditioning ischemia and then recovered in the presence of U0126. Bim expression was determined by immunoblot. Data shown are representative of three independent experiments.

To show that p42/p44 MAPK activation increases following preconditioning ischemia, antibodies to phosphorylated p42/p44 MAPK were used to probe immunoblots. The level of p42/p44 MAPK phosphorylation in control cells was low; however, phosphorylation of p42/p44 MAPK increased one hour following preconditioning ischemia. The increase in p42/p44 MAPK phosphorylation following preconditioning ischemia was blocked by U0126. In control cells treated with U0126 alone, there was no phosphorylation of MAPK. As a control, the same blot was probed for non-phospho-p42/p44 MAPK. Total levels of p42/p44 MAPK did not change following preconditioning ischemia or U0126 treatment. These data suggest that p42/p44 MAPK activity increased following preconditioning ischemia.

The effect of U0126 on Bim protein levels also was investigated following 30 minutes of OGD. A decrease in Bim protein expression was observed at one hour following 30 minutes of preconditioning ischemia. The decrease in Bim protein was blocked by U0126. However, U0126 had no effect on Bim protein expression in control-treated cells. These data suggest that the decrease in Bim levels observed following preconditioning is mediated via the p42/p44 MAPK system and that phosphorylation of Bim by MAPK is required for efficient Bim degradation.

### Example 2

### Interaction of Bim with TRIM2 from a Rat Brain Lysate

This example describes exemplary experiments performed to identify a Bim-interacting protein in a proteomics pull-down experiment and to confirm interaction of Bim polypeptide with TRIM2 protein from a rat brain lysate; see FIG. 10.

Proteomics pull-down experiments were performed using phosphorylated GST-Bim fusion protein and cell lysates prepared from rat cortical cell cultures, in order to identify potential Bim-interacting proteins with E3-ligase potential. Bim was phosphorylated by incubating GST-Bim with active p42 (Erk2) and p44 (Erk1) MAPK (60 minutes, 37° C.). (Phosphorylation was confirmed using anti phospho-Bim (Ser55) antibody (Stressgen).) The p42/p44 MAPK was removed by immunoprecipitation and confirmed by immunoblot (not shown). Phosphorylated GST-Bim was then bound to GST beads for 30 minutes, purified, and then incubated with lysate prepared from rat cortical cells (2 mg, 1 hour, 4° C.). Samples were loaded on a 10% SDS-polyacrylamide gel and subjected to electrophoresis. Gel fragments were cut out and analyzed by mass spectrometry. Peptide sequences were subjected to homology searching using the Prophet algorithm. A peptide was deemed identified if it had a prophet score of >0.9. The mass spectrometric data identified two known Bim binding proteins, dynein and a 14-3-3 isoform (14-3-3 zeta). Only one protein (TRIM2) with an E3-ligase motif (a RING or HECT domain) was identified out of 132 potential Bim-interacting proteins.

In order to confirm interaction of Bim and TRIM2, an immunoprecipitation experiment was performed in reverse to that used to generate the mass spectrometric data. An antibody against TRIM2 was agarose-immobilized and then incubated with 500 μg of rat brain lysate and GST-Bim. Immunoprecipitated material was then resolved by electrophoresis, blotted, and probed with an anti-GST antibody (Santa Cruz). FIG. 10 shows the resulting immunoblot, which reveals that GST-Bim was immunoprecipitated from rat brain lysates using a TRIM2 antibody. As a control the TRIM2 antibody was omitted from the reaction. Therefore, TRIM2 is a Bim-interacting protein.

### Example 3

### Interaction of Bim and TRIM2 in a Cell-Free Expression System

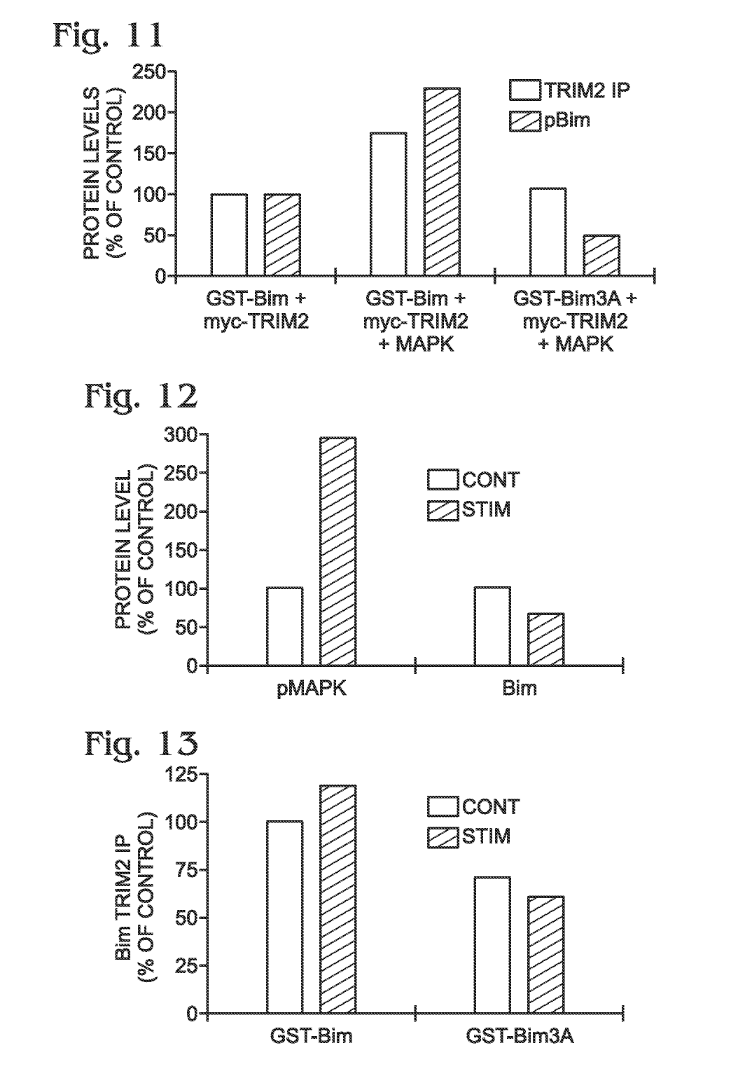

This example describes exemplary data that further confirms interaction of Bim and TRIM2 using a cell-free expression system; see FIG. 11.

An in vitro transcription-translation assay was employed to further confirm the binding of TRIM2 to Bim. Myc-tagged TRIM2 (myc-TRIM2) was synthesized by in vitro transcription of cDNA clones (pGBKT7-TRIM2) using T7 RNA polymerase, followed by in vitro translation in a nuclease-treated rabbit reticulocyte lysate system (Promega). Expression of myc-TRIM2 polypeptide was confirmed by immunoblot, which identified an 80 kDa polypeptide corresponding to a TRIM2 protein. Incubation of TRIM2 polypeptide with Bim resulted in low levels of Bim ubiquitination, however addition of active MAPK to the reaction enhanced Bim ubiquitination. Bim ubiquitination was present without adding MAPK, which may be due to high basal levels of active MAPK in the reticulocyte lysate supernatant.

To confirm further that Bim and TRIM2 interact, the translated myc-TRIM2 polypeptide was incubated with GST-Bim polypeptide (Millipore). Polypeptides were separated by GST immunoprecipitation and TRIM2 binding was determined by c-myc immunoblotting of immunoprecipitated proteins using an anti-c-myc antibody or with a phospho-specific Bim antibody against phosphor-Ser 65 of Bim. The results are quantified in FIG. 11. TRIM2 was immunoprecipitated much more efficiently when active p42/p44 MAPK (Millipore) was added to the reaction. To show that the precipitation was specific for phosphorylated Bim, TRIM2 also was incubated with the non-phosphorylatable mutant, GST-Bim3A. Precipitation of myc-TRIM2 with GST-Bim3A in the presence of MAPK was not observed. These data show that the binding of Bim protein to TRIM2 protein may require phosphorylation of Bim protein by p42/p44 MAPK on Ser 55, 65, 73, or a combination thereof.

### Example 4

### Interaction of Bim and TRIM2 in a MEF Cell Line

This example describes exemplary experiments demonstrating interaction of Bim and TRIM2 in a mouse embryonic fibroblast (MEF) cell line; see FIGS. 12 and 13.

Bim ubiquitination that is dependent on p42/p44 MAPK was originally described in the MEF cell line. To confirm that TRIM2 protein mediates Bim ubiquitination in the MEF cell line, cells were subjected to overnight serum starvation and then were stimulated with serum for one hour (“STIM”), which enhances Bim ubiquitination and degradation. Bim protein and phospho p42/p44 MAPK levels then were determined by immunoblot (FIG. 12). Compared to control (“CONT”), both an increase in MAPK phosphorylation and a decrease in Bim protein levels were observed in these cells following stimulation.

FIG. 13 shows quantified results from co-immunoprecipitation experiments analyzed by immunoblot with anti-GST antibody. MEF cell lysates were incubated with GST-Bim, and then were subjected to immunoprecipitation with anti-TRIM2 antibody. GST-Bim was observed to co-immunoprecipitate with TRIM2 protein from stimulated cell lysates, but not from unstimulated cell lysates. When MEF cell lysates were incubated with a nonphosphorylatable Bim mutant (GST-Bim3A), an increase in Bim immunoprecipitation was not observed with stimulation. These data show that in a cell line with well-characterized Bim ubiquitination, TRIM2 interacts with Bim in a MAPK-dependent manner.

### Example 5

### Interaction of Bim and TRIM2 in Neuronal Cultures with Ischemia

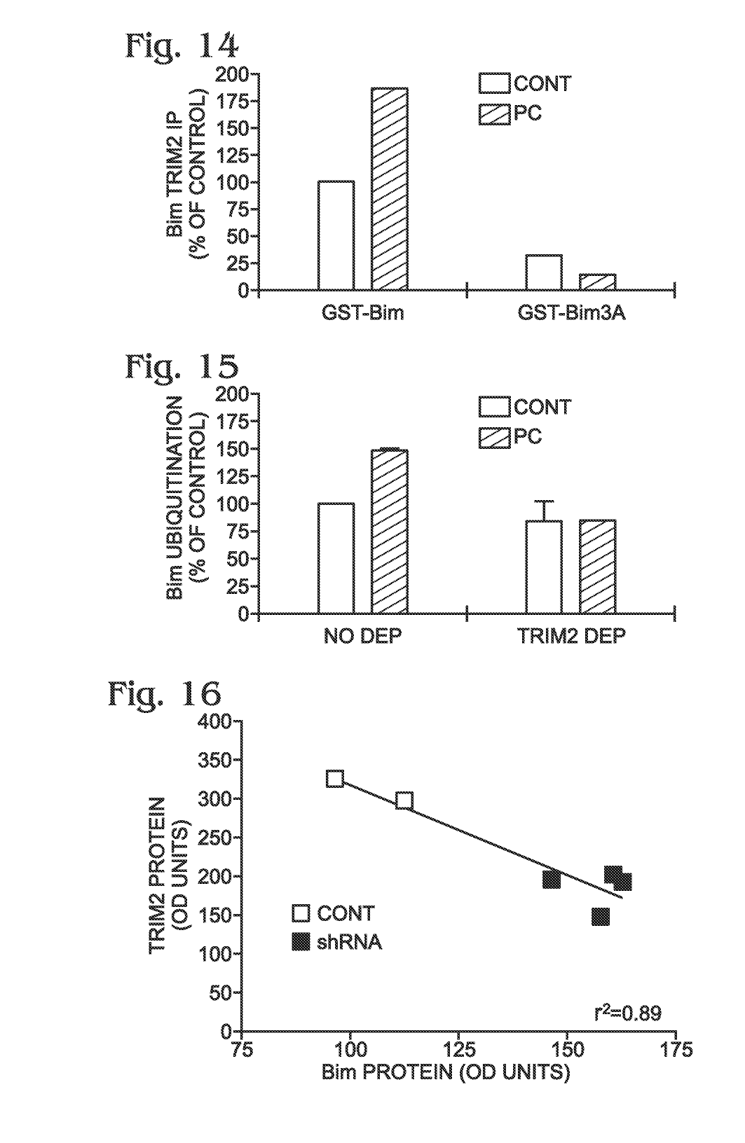

This example describes exemplary experiments demonstrating p42/p44 MAPK-dependent Bim and TRIM2 interaction under conditions that promote Bim ubiquitination in neurons; see FIGS. 14 and 15.

Preconditioning ischemia in cortical neurons was found to reduce Bim protein levels to less than 50% of control values (see Example 1). To follow up on this observation, co-immunoprecipitation was utilized to determine whether TRIM2 binding to Bim correlates with the degradation of Bim following preconditioning ischemia in neuronal cultures. GST-Bim was incubated with cell lysates from control neurons (“CONT”; open bars) and neurons exposed to preconditioning ischemia (“PC”; hatched bars). GST-Bim interacting with TRIM2 then was co-immunoprecipitated with anti-TRIM 2 antibody and detected by immunoblot with an anti-GST antibody.

FIG. 14 shows the results presented as a bar graph. Interaction of GST-Bim polypeptide with TRIM2 protein was found to be low in control cells, but increased one hour after a 30-minute period of preconditioning ischemia. Co-immunoprecipitation of Bim was not observed when lysates were incubated with a non-phosphorylatable Bim mutant, GST-Bim3A. Furthermore, co-immunoprecipitation of GST-Bim polypeptide with TRIM2 protein was reduced when cells were incubated with the MAPK kinase inhibitor U0126, which blocks Bim degradation and rapid ischemic tolerance (see Example 1 and data not shown).

FIG. 15 shows a bar graph presenting exemplary data on Bim ubiquitination in neuronal cell lysates. Ubiquitination of GST-Bim polypeptide was conducted by incubating this fusion protein in neuronal cell lysates, either with (“TRIM2 DEP”) or without (“NO DEP”) prior depletion of TRIM2 protein using an anti-TRIM2 antibody, and with lysates prepared from preconditioned neuronal cultures (“PC”; hatched bars) or control neuronal cultures (“CONT”; open bars). Bim ubiquitination was measured by immunoprecipitating GST-Bim from the lysates and then probing an immunoblot of immunoprecipitated GST-Bim for ubiquitin using an anti-ubiquitin antibody. The bar graph of FIG. 15 shows that immunodepletion of TRIM2 protein reduces Bim ubiquitination in lysates from preconditioned cells to that of control lysates. Taken together, these data confirm that under conditions where Bim ubiquitination increases, Bim binds to TRIM2 and removal of TRIM2 from cell lysates prevents Bim ubiquitination in an ex vivo assay following preconditioning ischemia.

### Example 6

### RNA Interference of TRIM2 Expression in Neurons

This example describes exemplary experiments demonstrating an inverse relationship between Bim and TRIM2 protein levels using knockdown of TRIM2 with a short hairpin RNA (shRNA); see FIG. 16.

If TRIM2 is the primary E3 ligase for Bim in cortical neurons, reduction of TRIM2 protein should increase Bim protein stability and thus the steady state level of Bim protein. In order to test this idea, shRNA directed to TRIM2 mRNA was employed to reduce TRIM2 protein expression.

FIG. 16 is a graph presenting exemplary data on TRIM2 and Bim protein levels in cortical neurons, with (“shRNA”) and without (“CONT”) RNA interference of TRIM2 protein expression. A lentivirus system was used to deliver a TRIM2 shRNA expression vector to the neurons by incubation with the neurons for 72 hours. TRIM2 and Bim protein levels were determined by immunoblot and are plotted in FIG. 16. Exposure of the neurons to TRIM2 shRNA resulted in a 40% decrease in TRIM2 protein levels. The shRNA produced only a modest decrease in TRIM2 expression, which may be explained by the relatively long half-life measured for TRIM2 protein, namely, a half-life exceeding 24 hours (not shown). Significantly, treatment with TRIM2 shRNA also produced a change in the ratio of TRIM2 to Bim protein, namely, a corresponding 40% increase in Bim protein levels in the same neurons, which is an inverse relationship and is consistent with TRIM2 protein functioning as a Bim E3-ligase.

### Example 7

### Bim/TRIM2 Expression Ratios in Breast Cancer Cell Lines

This example describes exemplary experiments suggesting a role of TRIM2 in regulating Bim degradation in breast tumor cells; see FIG. 17.

Intracellular mechanisms preventing apoptosis are frequently implicated in oncogenesis. While Bcl-2 is protective in ischemia, it may be harmful in cancer. Therefore, experiments were performed to determine whether TRIM2 overexpression and Bim degradation could play a role in breast cancer.

FIG. 17 shows a graph presenting exemplary data on Bim/TRIM2 protein ratios in established breast cancer cell lines. High levels of TRIM2 protein and low levels of Bim protein were observed in the breast tumor cell line HCC1419. In contrast, MCF-7 cells showed higher levels of Bim protein and lower levels of TRIM2 protein. Interestingly, HCC1419 cells overexpress HER2, whereas MCF-7 cells do not.

### Example 8

### Expression of TRIM2 and Bim in Breast Tumor Samples

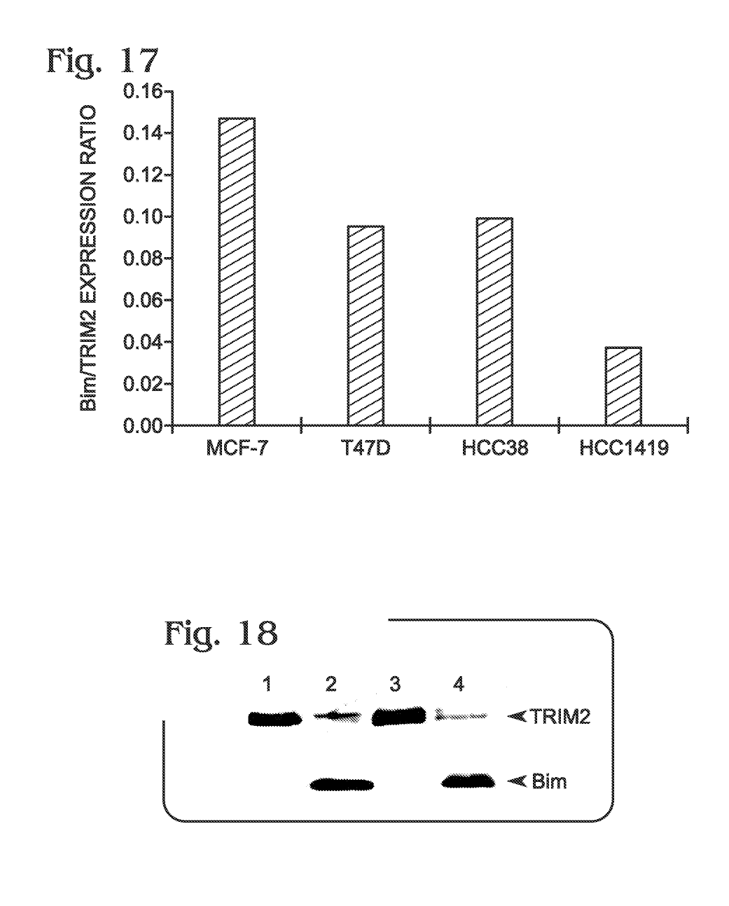

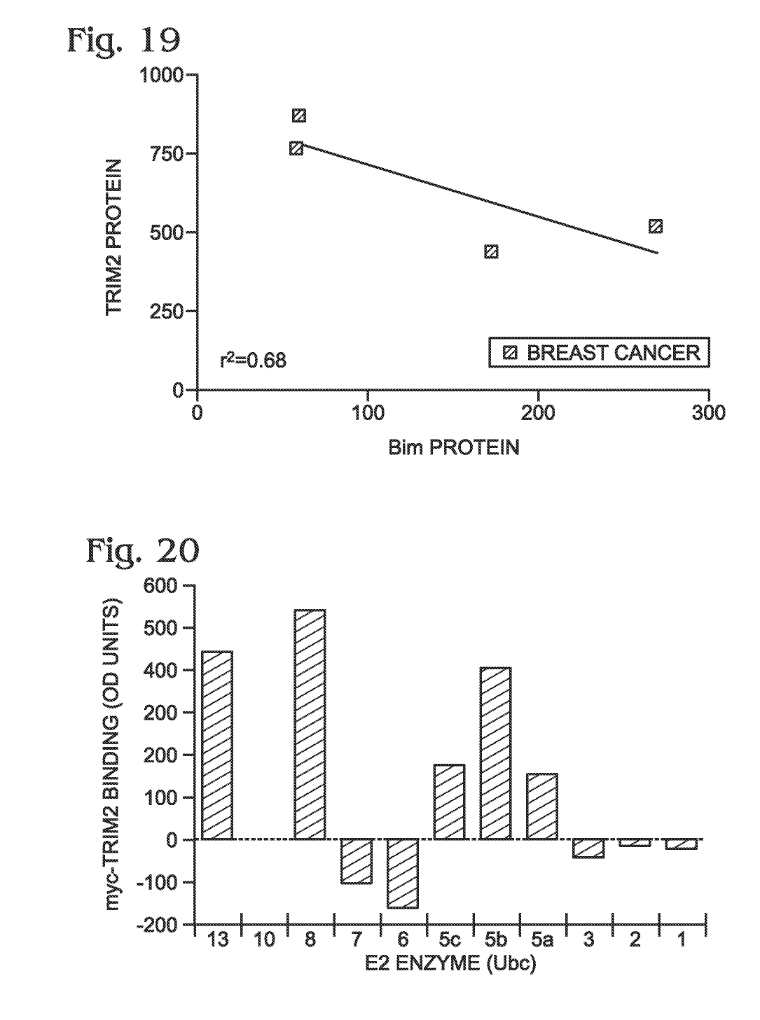

This example describes exemplary experiments analyzing expression of TRIM2 protein in primary breast tumors; see FIGS. 18 and 19.

FIG. 18 shows a pair of immunoblots presenting exemplary data on expression of TRIM2 and Bim protein in breast tumor samples, and FIG. 19 is a graph of the expression data of FIG. 18. Four samples of breast tissue were obtained during surgery to remove breast tumors. The samples were tested for Bim and TRIM2 protein expression levels using immunoblot. As shown in the figures, Bim and TRIM2 levels appear to be reciprocally regulated in these samples. Interestingly, samples 1 and 3, which are from patients with HER2/neu-positive tumors, show high levels of TRIM2. Samples 2 and 4, which were not overexpressing HER2/neu, showed lower TRIM2 levels and higher Bim levels. The expression of TRIM2 is unrelated to drug treatment, because all of these patients were untreated prior to surgery. These results suggest that Bim and TRIM2 protein expression may be altered in certain cancer types and particularly that TRIM2 protein expression may be raised in HER2/neu-positive, drug-naive tumors.

Additional experiments demonstrated that TRIM2 is upregulated in other cancer types. Bim and TRIM2 appear reciprocally regulated in lung, muscle, and sarcoma tumor samples. This suggests that TRIM2 may play a role in tumor resilience to cell death in other types of cancer.

### Example 9

### Interaction of TRIM2 with E2 Ubiquitin-Conjugating Enzymes

This example describes exemplary experiments demonstrating interaction of TRIM2 with E2 ubiquitin-conjugating enzymes; see FIG. 20.

FIG. 20 show a bar graph presenting exemplary data on interaction of TRIM2 polypeptide with various E2 ubiquitin-conjugating enzymes. E2 ubiquitin-conjugating enzymes supply the ubiquitin to the E3-ligase for conjugation to a target protein. To test interaction of TRIM2 polypeptide with E2 enzymes, a set of E2 enzymes, namely, Ubc 1, 2, 3, 5a, 5b, 5c, 6, 7, 8, 10, and 13, were loaded onto a gel and transferred onto a PVDF membrane. The immobilized proteins were incubated with E1 enzyme, ubiquitin, and ATP for 10 min, and then with c-myc-TRIM2, GST-Bim and active ERK1/2 (MAPK) for 20 min at room temperature. The membrane was washed, probed with anti-c-myc antibody, exposed, and quantified. The results are present in graphical form in FIG. 20. TRIM2 was bound to Ubc 5a, 5b, 5c, 8, and 13 which suggests that these E2 enzymes can supply the ubiquitin to TRIM2 for ubiquitination of target proteins.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A method of drug screening, comprising:
forming an assay mixture including a Bim polypeptide, a TRIM2 polypeptide, and one or more test compounds;
detecting interaction of the Bim and TRIM2 polypeptides with each other; and
determining whether the one or more test compounds affect the interaction.

2. The method of claim 1, further comprising a step of selecting at least one test compound as a candidate drug for treatment of a disease characterized by an abnormal amount of cell death, based at least in part on the step of determining.

3. The method of claim 1, wherein the step of selecting selects at least one test compound that inhibits the interaction, such that the at least one test compound is a candidate drug for treating cancer by increasing cell death.

4. The method of claim 1, wherein the step of selecting selects at least one test compound that enhances the interaction, such that the at least one test compound is a candidate drug for reducing cell death associated with neurological disease.

5. The method of claim 1, wherein at least one of the Bim and TRIM2 polypeptides has a luminescent tag, and wherein the step of detecting includes a step of optically detecting a signal that indicates an amount of interaction of the polypeptides.

6. The method of claim 5, wherein the step of optically detecting a signal includes a step of measuring fluorescence resonance energy transfer or fluorescence polarization.

7. The method of claim 5, wherein the Bim and TRIM2 polypeptides include a respective Bim portion and TRIM2 portion each conjugated to a distinct fluorescent polypeptide.

8. The method of claim 1, further comprising a step of contacting one or both of the Bim and TRIM2 polypeptides with an antibody that recognizes only one of the polypeptides.

9. The method of claim 8, wherein at least one of the Bim and TRIM2 polypeptides includes an epitope tag.

10. The method of claim 1, further comprising a step of phosphorylating the Bim polypeptide before the step of detecting interaction.

11. The method of claim 10, further comprising a step of contacting the Bim polypeptide with a MAP kinase.

12. The method of claim 1, wherein the step of forming an assay mixture includes a step of introducing an inhibitor of MAPK kinase.

13. The method of claim 1, wherein the step of forming an assay mixture is repeated with different test compounds in distinct wells of a microplate.

14. The method of claim 1, wherein the step of detecting interaction is repeated without the one or more test compounds in the assay mixture, and wherein the step of determining is based on a comparison of the interaction detected with and without a test compound.

15. The method of claim 1, wherein the one or more test compounds include a polypeptide identical or corresponding to only a fragment of Bim.

16. The method of claim 1, wherein the assay mixture includes biological cells that express the Bim and TRIM2 polypeptides.

17. A composition for drug screening, comprising:
an engineered Bim polypeptide; and
an engineered TRIM2 polypeptide that interacts with the Bim polypeptide.

18. The composition of claim 17, wherein the Bim and TRIM2 polypeptides include a respective Bim portion and TRIM2 portion each conjugated to a distinct fluorescent polypeptide, and wherein the fluorescent polypeptides are configured to permit interaction of the Bim and TRIM2 polypeptides to be detected by fluorescence resonance energy transfer.

19. A method of treating cancer, comprising:
testing one or more samples from a cancer patient for levels of TRIM2 protein, Bim protein, and active MAP kinase protein; and
treating the cancer patient with at least one drug that inhibits degradation of Bim protein if the level of Bim protein is below a first threshold, the level of TRIM2 protein is above a second threshold, and the level of active MAP kinase protein is above a third threshold.

20. The method of claim 19, wherein the at least one drug includes a MAPK kinase inhibitor, a proteasome inhibitor, or both.
